(19) United States
(12) Patent Application Publication
(10) Pub. No.: US 2004/0265956 A1
(43) Pub. Date: Dec. 30, 2004

(54) Method for producing target substance by fermentation

(76) Inventors: Rie Takikawa, Kawasaki (JP); Akira Imaizumi, Kawasaki (JP); Kazuhiko Matsui, Kawasaki (JP); Hiroyuki Kojima, Kawasaki (JP)

Correspondence Address: AJINOMOTO CORPORATE SERVICES, LLC, INTELLECTUAL PROPERTY DEPARTMENT, 1120 CONNECTICUT AVE., N.W., WASHINGTON, DC, 20036, US

(21) Appl. No.: 10/701,923

(22) Filed: Nov. 6, 2003

(30) Foreign Application Priority Data
Nov. 11, 2002 (JP) 2002-327205

(51) Int. Cl. C12P 13/04; C12P 13/08; C12Q 1/68; C07H 21/04; C12N 15/74

(52) U.S. Cl. 435/69.1; 435/106; 435/488; 435/252.33; 536/23.7; 435/115

(57) ABSTRACT

The present invention describes a method for producing a target substance using a microorganism comprising culturing an Escherichia bacterium in a medium to produce and accumulate the target substance in the medium or the bacterium, and collecting the target substance. More specifically, the bacterium of the present invention can be a strain in which the fis gene on bacterial chromosome is disrupted so that the FIS protein does not function normally in the bacterium.

### BACKGROUND OF THE INVENTION

1. Field of the Invention

The present invention relates to a method for producing a target substance such as an L-amino acid by fermentation utilizing a microorganism. More specifically, the present invention relates to a method for producing a target substance in a microorganism whereby the fis gene is disrupted. The present invention is useful in the fermentation industry.

2. Description of the Related Art

Chromosome DNA of Escherichia coli is folded into a nucleosome-like structure called a nucleoid by two or more of DNA-binding proteins. Such proteins are called nucleoid-structuring proteins.

FIS (factor for inversion stimulation) is one of the major nucleoid-structuring proteins and is encoded by the fis gene. The fis gene exists at a position of 73.4 min on the Escherichia coli chromosome. Fis gene expression is induced in the growth phase and suppressed in the stationary phase. It has been reported that if the fis gene of an Escherichia coli strain is disrupted, the growth rate decreases as compared to a wild-type strain, even in an extremely nutrient-rich medium. However, if the culture is conducted in a limited-growth medium, the fis-gene-disrupted stain grows at the same rate as that of a wild-type strain (Nilsson et al., The Journal of Biological Chemistry, Vol. 269, 9460-9465, 1994). Normal production of amino acids is usually conducted in a limited-growth medium so as to suppress cell growth. Furthermore, FIS is a global transcription regulating factor which positively or negatively regulates expression of two or more kinds of genes, and it has been reported that FIS regulates expression of transfer RNA and ribosome RNA (Nilsson et al., The EMBO Journal, Vol. 9, 727-734, 1990) as well as expression of genes involved in metabolism and so forth (Xu et al., Journal of Bacteriology, Vol. 177, 938-947,1995).

A nucleoid-structuring protein known to be induced in the growth phase and known to positively or negatively regulate expression of two or more kinds of genes like FIS includes H-NS (Hulton et al., Cell, Vol. 63, 631-642, 1990). H-NS is encoded by the hns gene, which exists at a position of 27.8 min on the Escherichia coli chromosome.

Examples of other major nucleoid-structuring proteins include, for example, HU, DPS and so forth. HU is a heterodimer consisting of Huα and Huβ encoded by the hupA and hupB genes, which exist at 90.5 min and 9.7 min, respectively, on the Escherichia coli chromosome (Wada et al., Journal of Molecular Biology, Vol. 204,581-591, 1988). Expression of these genes is observed in both of the growth phase and the stationary phase. DPS is encoded by the dps gene, which exists at 18.3 min on the Escherichia coli chromosome. Its expression is suppressed in the growth phase and induced in the stationary phase (Almion et al., Genes and Development, Vol. 6,2646-2654, 1992).

To date, there have been no reports about improved production of substances by regulating expression of genes coding for nucleoid-structuring proteins such as the fis, hns, hupAB and dps genes.

### SUMMARY OF THE INVENTION

An object of the present invention is to improve production efficiency and/or production rate in the production of useful substances by fermentation using bacterium belonging to the genus Escherichia.

It is an object of the present invention to provide a method for producing a target substance using a bacterium belonging to the genus Escherichia comprising culturing said bacterium in a medium resulting in production and accumulation of said target substance in said medium or said bacterium, and collecting said target substance, wherein said bacterium has an ability to produce said target substance, and FIS protein does not function normally in said bacterium.

It is a further object of the present invention to provide a method as above, wherein said fis gene has been disrupted so that it does not function normally.

It is a yet a further object of the present invention to provide the method as described above, wherein said bacterium belonging to the genus Escherichia is Escherichia coli.

It is a still a further object of the present invention to provide a method as described above, wherein said target substance is selected from the group consisting of an L-amino acid and a protein.

It is a further object of the present invention to provide the method as described above, wherein said target substance is an L-amino acid.

It is a further object of the present invention to provide the method as described above, wherein said L-amino acid is L-lysine.

### BRIEF DESCRIPTION OF THE DRAWINGS

FIG. 1 shows growth patterns of the strains MG1655, MG1655Δfis, MG1655Δhns, MGΔdps and MGΔhupAB.

FIG. 2 shows growth patterns of the WC196 and WC196Δfis.

FIG. 3 shows glucose consumption ferns of the strains WC196 and WC196Δfis strains.

FIG. 4 shows lysine accumulation patterns of the strains WC196 and WC196Δfis.

FIG. 5 shows growth-patterns of the strains WC196/pCABD2 and WC196Δfis/pCABD2.

FIG. 6 shows glucose consumption patterns of the strains WC196/pCABD2 and WC196Δfis/pCABD2.

FIG. 7 shows lysine accumulation patterns of the strains WC196/pCABD2 and WC196Δfis/pCABD2.

### DESCRIPTION OF THE PREFERRED EMBODIMENTS

The inventors of the present invention assiduously studied in order to achieve the foregoing objects. As a result, it was found that substance production by Escherichia bacteria could be improved by modifying a gene coding for a nucleoid-structuring protein which universally exists in Escherichia bacteria Specifically, it was found that an ability to produce a target substance could be improved by disrupting the fis gene in Escherichia bacteria.

The bacterium belonging to the genus Escherichia used in the present invention is not particularly limited so long as it is a microorganism belonging to the genus Escherichia and has an ability to produce a target substance. Specifically, bacterium disclosed in Neidhardt et al. (Neidhardt, F. C. et al., Escherichia coli and Salmonella Typhimurium, American Society for Microbiology, Washington D.C., 1208, Table 1) is encompassed by the present invention. More specifically, the bacterium useful in the present invention includes, but is not limited to Escherichia coli.

The “ability to produce a target substance” is defined as an ability to produce the target substance in an amount which is collectable from cells or a medium when the Escherichia bacterium used in the present invention is cultured in the medium. Preferably, it means an ability to produce the target substance in a larger amount than wild-type or otherwise unmodified strains of the Escherichia bacterium.

The target substance is not particularly limited so long as it can be produced by a bacterium belonging to the genus Escherichia. Examples of such target substance include various L-amino acids such as L-lysine, L-threonine, L-homoserine, L-glutaric acid, L-leucine, L-isoleucine, L-valine and L-phenylalanine, proteins (including peptides), nucleic acids such as guanine, inosine, guanylic acid and inosinic acid, vitamins, antibiotics, growth factors, physiologically active substances and so forth, which have been conventionally produced by using Escherichia bacteria. Furthermore, the present invention may be applied even to those substances that have not been produced to date by using bacteria belonging to the genus Escherichia.

The target substance may be a L-amino acid from the aspartic acid family of amino acids. This family includes L-lysine, L-threonine, and L-methionine.

Examples of L-lysine producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine coexists in a medium. Examples of the L-lysine analogue include, but are not limited to, oxalysine, lysine hydroxamate, S-(2-aminoethyl)-L-cysteine (AEC), γ-methyllysine, α-chlorocaprolactam and so forth. Mutants having resistance to these lysine analogues can be obtained by subjecting bacteria belonging to the genus Escherichia to a conventional artificial mutagenesis treatment. Specific examples of bacterial stains useful for producing L-lysine include Escherichia coli AJ11442 (FERM BP-1543, NRRL B-12185; see Japanese Patent Laid-open Publication (Kokai) No. 56-18596 and U.S. Pat. No. 4,346,170) and Escherichia coli VL611. In these microorganisms, feedback inhibition of aspartokinase by L-lysine is desensitized.

In addition to the above, L-threonine producing bacteria is encompassed since inhibition of aspartokinase by L-lysine is generally desensitized also in L-threonine producing bacteria

In the examples described herein, the strain WC196 is used as a L-lysine producing bacterium of Escherichia coli. This bacterial strain was bred by conferring AEC resistance to the stain W3110, which was derived from Escherichia coli K-12. The resulting stain was designated as the Escherichia coli AJ13069 strain, and was deposited at the National Institute of Bioscience and Human-Technology, Agency of Industrial Science and Technology (currently National Institute of Advanced Industrial Science and Technology, Intentional Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was transferred to an international depository under the provisions of the Budapest Treaty on Sep. 29, 1995, and received an accession number of FERM BP-5252 (see International Patent Publication WO96/17930).

Examples of L-threonine producing bacteria belonging to the genus Escherichia include, but are not limited to, Escherichia coli VKPM B-3996 (RIA 1867) (see U.S. Pat. No. 5,175,107), MG442 strain (see Gusyatiner et al., Genetika (in Russian), 14, pp.947-956,1978) and so forth

Examples of L-homoserine producing bacteria belonging to the genus Escherichia include, but are not limited to, the strain NZ10, which is a Leu+ revertant of the strain C600 (see Appleyard R. K., Genetics, 39, pp.440-452, 1954).

Examples of L-glutamic acid producing bacteria belonging to the genus Escherichia include, but are not limited to, the AJ12624 strain (FERM BP-3853, see French Patent Laid-open Publication No. 2,680,178), Escherichia coli B 11, Escherichia coli K-12 (ATCC10798), Escherichia coli B (ATCC11303) and Escherichia coli W (ATCC9637).

Examples of L-leucine producing bacteria belonging to the genus Escherichia include bacterial stains having β-2-thienylalanine resistance, bacterial strains having β-2-thienylalanine resistance and β-hydroxyleucine resistance (see Japanese Patent Publication (Kokoku) No. 62-34397 for the above) and bacterial strains having 4-azaleucine resistance or 5,5,5-trifluoroleucine resistance (see Japanese Patent Laid-open Publication (Kokai) No. 8-70879). Specifically, there can be mentioned the strain AJ11478 (FERM P-5274, see Japanese Patent Publication (Kokoku) No. 62-34397).

Examples of L-isoleucine producing bacteria belonging to the genus Escherichia include, but are not limited to, Escherichia coli KX141 (VKPM B-4781, see European Patent Laid-open Publication No. 519,113).

Examples of L-valine producing bacteria belonging to the genus Escherichia include, but are not limited to, Escherichia coli VL1970 (VKPM B-4411, see European Patent Laid-open Publication No. 519,113).

Examples of L-phenylalanine producing bacteria include, but are not limited to, Escherichia coli AJ12604 (FERM BP-3579, see European Patent Laid-open Publication No. 488,424).

Furthermore, bacteria belonging to the genus Escherichia having L-amino acid producing ability can also be bred by introducing DNA having genetic information involved in biosynthesis of L-amino acids, as well as enhancing the L-amino acid producing ability by utilizing a gene recombination technique. For example, genes that can be introduced into L-lysine producing bacteria include, but are not limited to, genes which encode for enzymes of the biosynthetic pathway of L-lysine such as phosphoenolpyruvate carboxylase, aspartokinase, dihydrodipicolinate synthetase, dihydrodipicolinate reductase, succinyldiaminopimelate transaminase and succinyldiaminopimelate deacylase. In the case of a gene encoding an enzyme which suffers from feedback inhibition by L-aspartic acid or L-lysine, such as phosphoenolpyruvate carboxylase, aspartokinase, and/or dihydrodipicolinate synthetase, it is desirable to use a mutant gene which encodes an enzyme in which such inhibition is desensitized.

Furthermore, examples of genes that can be introduced into L-glutamic acid producing bacteria include, but are not limited to, genes which encode for glutamate dehydrogenase, glutamine synthetase, glutamate synthase, isocitrate dehydrogenase, aconitate hydratase, citrate synthase, phosphoenolpyruvate carboxylase, pyruvate dehydrogenase, pyruvate kinase, phosphoenolpyruvate synthase, enolase, phosphoglyceromutase, phosphoglycerate kinase, glyceraldehyde-3-phosphate dehydrogenase, triose phosphate isomerase, fructose bis-phosphate aldolase, phosphofructokinase, glucose phosphate isomerase and so forth.

Examples of genes that can be introduced into L-valine producing bacteria include, but are not limited to, an ilvGMEDA operon, preferably, an ilvGMEDA operon that does not express threonine deaminase activity and in which attenuation is cancelled (see Japanese Patent Laid-open Publication (Kokai) No. 847397).

Furthermore, an activity of an enzyme that catalyzes a reaction for producing a compound other than the target L-amino acid by branching off from the biosynthetic pathway of the L-amino acid may be decreased or made deficient. For example, enzymes that catalyze a reaction for producing a compound other than L-lysine by branching off from the biosynthetic pathway of L-lysine include homoserine dehydrogenase (refer to International Patent Publication WO95/23864). Furthermore, enzymes that catalyze a reaction for producing a compound other than L-glutamic acid by branching off from the biosynthetic pathway of L-glutamic acid include, but are not limited to, α-ketoglutarate dehydrogenase, isocitrate lyase, phosphate acetyltransferase, ac et kinase, acetohydroxy acid synthase, acetolactate synthase, formate acetyltransferase, lactate dehydrogenase, glutamate decarboxylase, 1-pyrroline dehydrogenase and so forth.

Furthermore, bacteria belonging to the genus Escherichia having an ability to produce a nucleic acid are described in detail in, for example, International Patent Publication WO99/03988. More specifically, a description of Escherichia coli FADRaddG-8-3::KQ strain (purFKQ, purA⁻, deoD⁻, purR⁻, add⁻, gsk⁻) is included in that publication. This strain has the ability to produce inosine and guanosine. This strain contains a mutant purF gene coding for PRPP amidotransferase in which the lysine residue at a position of 326 is replaced with a glutamine residue, and feedback inhibition by AMP and GMP is desensitized. Furthermore, in this strain, the succinyl AMP synthase gene (purA), purine nucleoside phosphorylase gene (deoD), purine repressor gene (purR), adenosine deaminase gene (add) and inosine-guanosine kinase gene (gsk) are disrupted. This strain was given a private number of AJ13334, and deposited at the National Institute of Bioscience and Human-Technology, Agency of Industrial Science and Technology, Ministry of International Trade and Industry (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Jun. 24, 1997 as an international deposit under the provisions of the Budapest Treaty and received an accession number of FERM BP-5993.

Furthermore, the present invention includes proteins that can be produced by a genetic engineering method, and specifically include, but are not limited to acid phosphatase and GFP (Green Fluorescent Protein) and the like.

In addition, bacteria belonging to the genus Escherichia having the ability to produce useful substances such as other L-amino acids, proteins (including peptides), nucleic acids, vitamins, antibiotics, growth factors and physiologically active substances can also be used for the present invention.

In the breeding of bacteria belonging to the genus Escherichia having such a target substance producing ability as mentioned above, the present invention includes introduction of a gene into Escherichia bacteria to enhance their ability. For this purpose, a method can be used in which a vector which is autonomously replicable in a cell of bacterium belonging to the genus Escherichia is ligated to the gene to construct recombinant DNA, and Escherichia coli is transformed with it. In addition, it is also possible to incorporate a target gene into the host chromosome by using a method such as transduction, transposon (Berg, D. E. and Berg, C. M., Bio/Technol. 1, p.417,1983), Mu phage, (Japanese Patent Laid-open Publication (Kokai) No. 2-109985) or homologous recombination (Experiments in Molecular Genetics, Cold Spring Harbor Lab., 1972).

The Escherichia bacterium used in the present invention is a bacterium having the aforementioned ability to produce a target substance in which the FIS protein does not normally function in the cell. The expression “FIS protein does not normally function” is defined either as a state such that transcription or translation of the fis gene is decreased, and thus the amount of FIS protein, or the gene product thereof, is not produced or is produced in a decreased amount, or a state such that a mutation occurs in the produced FIS protein, and thus the original function of the FIS protein is degraded or lost. Typical examples of the Escherichia bacterium in which the FIS protein does not normally function include, but are not limited to, a gene-disrupted strain in which the fis gene on the chromosome is disrupted by a gene recombination technique, and a mutant strain in which the functional FIS protein is no longer produced due to a mutation that occurs in the expression regulatory sequence or the coding region of the fis gene on the chromosome.

Examples of the nucleotide sequence of the fis gene of Escherichia coli (sequence of the nucleotide numbers 1067 to 1363 in the nucleotide sequence of the GenBank accession number AE000405) and the amino acid sequence of the FIS protein are shown as SEQ ID NOS: 21 and 22, respectively.

In the present invention, the “FIS protein” may be, besides the FIS protein of Escherichia coli having the amino acid sequence of SEQ ID NO: 22, a homologue of this protein. Furthermore, the fis gene to be disrupted may be, besides the fis gene of Escherichia coli having the nucleotide sequence of SEQ ID NO: 21, a homologue of this gene. For example, the homologue of the fis gene includes a gene which is hybridizable with a probe having the nucleotide sequence of SEQ ID NO: 21 or a some part thereof under stringent conditions, and codes for a protein having the function of the FIS protein. The “stringent conditions” referred to herein are defined as conditions under which a so-called specific hybrid is formed, and a non-specific hybrid is not formed. Although it is difficult to clearly express this condition by using any numerical value, for example, the stringent conditions include conditions under which DNA having high homology, for example, DNA having homology of 50/o or more, preferably 70% or more, more preferably 900/o or more, still more preferably 95% or more will hybridize with each other, but DNA having homology lower than the above will not hybridize with each other. Alternatively, the stringent conditions are exemplified by conditions under which DNA are hybridized with each other at a salt concentration which corresponds to typical conditions of washing for Southern hybridization, for example, 1×SSC, 0.1% SDS, and preferably 0.1×SSC, 0.1% SDS, at 60° C.

A part of the nucleotide sequence of SEQ ID NO: 21 can also be used as a probe. Such a probe can be produced by PCR using oligonucleotides based on the nucleotide sequence of SEQ ID NO: 21 as primers, and a DNA fragment including the nucleotide sequence of SEQ ID NO: 21 as a template. When a DNA fragment having a length of about 300 bp is used as the probe, 2×SSC, 0.1% SDS at 50° C. can be mentioned as the condition of washing for hybridization.

The same gene as the fis gene on the chromosome of target Escherichia bacterium is preferably used as the fis gene in the gene destruction described below. However, a gene having homology to such a degree that homologous recombination in a cell should be possible can also be used.

Hereinafter, an example of the method for disrupting the fis gene on the chromosome by a gene recombination technique will be explained. The fis gene on the chromosome can be disrupted by transforming an Escherichia bacterium with DNA including a fis gene modified so as not to produce FIS which normally functions by deleting a part of the fis gene (deletion-type fis gene) and allowing recombination between the deletion-type fis gene and the fis gene on the chromosome. Such gene disruption by homologous recombination has already been established, and there is a method using linear DNA, a method using a plasmid including a temperate sensitive replication control region, and so forth. The method using a plasmid including a temperature sensitive replication control region is preferred due to its reliability.

The fis gene on the host chromosome can be replaced with the deletion-type fis gene as follows. For example, recombinant DNA can be prepared by ligating a temperature sensitive replication control region, a mutant fis gene and a marker gene showing resistance to a drug such as ampicillin. Then, a bacterium belonging to the genus Escherichia is transformed with this recombinant DNA, and the transformant strain is cultured at a temperature at which the temperature sensitive replication control region does not function. Then, the bacterium is further cultured in a medium containing the drug to obtain a transformant strain in which the recombinant DNA is incorporated into the chromosomal DNA.

Recombination of the chromosomal fis gene and the newly inserted recombinant DNA occurs in the strain when inserted as described above. As a result, two fusion genes containing the chromosome fis gene and the deletion-type fis gene are inserted into the chromosome on the both sides of the other part of the recombinant DNA, i.e. the vector portion, temperature sensitive replication control region and drug resistance marker. Therefore, the transformant strain expresses a normal FIS protein, since the normal fis gene is dominant in this state.

Subsequently, in order to maintain only the deletion-type fis gene on the chromosomal DNA, one copy of the fis gene is eliminated from the chromosome DNA along with the vector region (including the temperature sensitive replication control region and the drug resistance marker) by recombination of two of the fis genes. At this time, there is the case where the normal fis gene is left on the chromosomal DNA and the deletion-type fis gene is eliminated, or conversely, the case where the deletion-type fis gene is left on the chromosomal DNA and the normal fis gene is eliminated. In either case, the eliminated DNA is harbored in the cell in the form of a plasmid when the strain is cultured at a temperature where the temperature sensitive replication control region functions. On the other hand, if the strain is cultured at a temperature where the temperature sensitive replication control region does not function, a plasmid containing the normal fis gene is removed from the cell when the deletion-type fis gene is left on the chromosome DNA. Therefore, by confirming the structure of the fis gene in the cell by colony PCR or the like, there can be obtained a strain containing the deletion-type fis gene on the chromosome DNA and removal from the cell of the normal fis gene.

pMAN997 (International Patent Publication WO99/03988) is one example of a plasmid having a temperature sensitive replication control region that functions in a cell of bacterium belonging to the genus Escherichia. This plasmid is used in the examples described herein

Techniques used for usual gene recombination such as digestion and ligation of DNA, transformation, extraction of recombinant DNA from a transformant strain and PCR are described in detail in references well known to those skilled in the art, for example, Sambrook, J., Fritsch, E. F., Maniatis, T., Molecular Cloning, Cold Spring Harbor Laboratory Press, 1989 and so forth

Furthermore, a mutant strain in which the FIS protein no longer functions can be obtained by treating a bacterium belonging to the genus Escherichia by ultraviolet radiation or with a mutagenesis agent used for a conventional mutation treatment such as N-methyl-N′-nitro-N-nitrosoguanidine (NTG) or nitrous acid.

The target substance can be produced by culturing an Escherichia bacterium obtained as described herein. The Escherichia bacterium used to produce the target substance has a target substance-producing ability and an FIS protein which does not function normally. The Escherichia bacterium produces the target substance, resulting in the accumulation of the target substance in the medium or in the bacterium. The target substance may be collected from either the bacterium or the medium. In the present invention, the production rate or production efficiency of the target substance can be improved by using an Escherichia bacterium having the aforementioned properties. It is believed that this is because the fis gene expressed in wild-type strains of Escherichia bacteria results in the acceleration or inhibition of expression of a group of genes (including known and unknown genes) regulated by the FIS protein, whereas expression of the aforementioned group of genes is not regulated in a strain in which a normal FIS protein does not function normally.

Conventionally-used well known media can be used as the medium for culture of bacteria belonging to the genus Escherichia in the present invention, depending on the bacterial strain or the target substance. That is, usual media containing a carbon source, nitrogen source, inorganic ion and other organic components as require can be used. No special medium for carrying out the present invention is required

Sugars such as glucose, lactose, galactose, fructose and starch hydrolysate, and alcohols such as glycerol and sorbitol, and organic acids such as fumaric acid, citric acid and succinic acid and so forth can be used as the carbon source in the present invention

Inorganic ammonium salts such as ammonium sulfate, ammonium chloride and ammonium phosphate, and organic nitrogen such as soybean hydrolysate, ammonia gas, aqueous ammonia and so forth can be used as the nitrogen source in the present invention.

It is preferable to add required substances such as vitamin B1, L-homoserine and L-tyrosine, yeast extract and so forth as an organic trace nutrient in suitable amounts depending on the properties of Escherichia bacteria. In addition to these substances, small amounts of potassium phosphate, magnesium sulfate, iron ion, manganese ion and so forth can be added as required

The culture may be performed under well-known conditions that are conventionally used depending on the bacterial strain employed. For example, culture is preferably performed under an aerobic condition for 16-120 hours. The culture temperature is controlled to be 25-45° C. and pH is controlled to be 5-8 during the culture. Inorganic or organic acidic or alkaline substances as well as ammonia gas and so forth can be used to adjust the pH.

Once the culture is completed, collection of the target substance from the medium or the bacterium requires no special method for the present invention. That is, collection of the target substance can be attained by a combination of well-known methods, for example, methods using an ion exchange resin, precipitation and others depending on the target substance. Furthermore, target substance which has accumulated in the bacterium can be collected from cell extraction methods or membrane fractionation methods depending on the target substance, after physically or enzymatically disrupting the bacteria. Depending on the target substance, the target substance can be utilized as a microbial catalyst or the like while it exists in bacteria

According to the sent invention, production rate or production efficiency can be improved for useful substances such as L-amino acids by using Escherichia bacteria.

### EXAMPLES

Hereinafter, the present invention will be more specifically explained with reference to the following examples.

### Example 1

### Disruption of Gene Coding for Nucleoid-Structuring Protein Of Escherichia Coli MG1655 and its Effect on Growth

A gene coding for a nucleoid-structuring protein of Escherichia coli was disrupted by crossover PCR (see Link, A. J., Phillips, D., Church, G M., J. Bacteriol., Vol. 179, 6228-6237,1997).

(1) Disruption of fis Gene

The oligonucleotides of SEQ ID NOS: 1 and 2 (Primers 1 and 2) were synthesized as primers for amplifying a region of about 300 bp including about 20 bp at the N-terminus of the coding region of the fis gene and an upstream region of the same. The oligonucleotides of SEQ ID NOS: 3 and 4 (Primers 3 and 4) were synthesized as primers for amplifying a region of about 300 bp including about 20 bp at the C-terminus of the coding region of the fis gene and a downstream region of the same. Primers 2 and 3 included common sequences complementary to each other as parts thereof, and the primers were designed so that a part of ORF of the fis gene should be deleted when the amplification product was ligated at those portions.

First, PCR was performed by using combinations of Primers 1 and 2 and Primers 3 and 4 and genomic DNA of a wild-type strain MG1655 prepared by a usual method as a template. In this PCR, Primers 1 and 2 and Primers 4 and 3 were used in a molar ratio of 10:1. Secondly, PCR was performed by using the obtained product of the first PCR as a template and Primers 1 and 4. A DNA fragment including the deletion-type fis gene constructed by the second PCR was cloned in pGEMT-Easy (a cloning vector kit produced by Promega) according to the protocol to obtain a recombinant vector pGEM-fis.

pGEM-fis was digested with EcORI to obtain a DNA fragment including the deletion-type fis gene. This digested fragment was ligated to a temperature sensitive plasmid pMAN997 (see International Patent Publication WO99/03988) digested with the same enzyme and purified by using DNA ligation Kit Ver. 2 (Takara Shuzo). The aforementioned pMAN997 was obtained by exchanging the VspI-HindIII fragments of pMAN031 (J. Bacteriol., 162, 1196(1985)) and pUC19 (Takara Shuzo).

Escherichia coli JM109 competent cells (Takara Shuzo) were transformed with the aforementioned ligation reaction mixture, inoculated on an LB agar plate containing 25 μg/ml of ampicillin (Meiji Seika) (LB+ampicillin) and cultured at 30° C. to select ampicillin resistant colonies. The colonies were cultured in the LB medium containing 25 μg/ml of ampicillin in test tubes at 30° C., and plasmids were extracted from the cells by using Wizard Plus Miniprep (Promega). These plasmids were digested with EcoRI, and a plasmid containing a fragment of a target length was selected as a plasmid pMANΔfis for fis disruption.

Escherichia coli MG1655 was transformed by using pMANΔfis.

The transformant strains were cultured on LB+ampicillin plates at 30° C., and ampicillin resistant colonies were selected. The selected colonies were cultured overnight at 30° C. as liquid culture, diluted 10³ times and inoculated on LB+ampicillin plates, and ampicillin resistant colonies were selected at 42° C. At this stage, pMANΔfis was incorporated into the chromosome DNA.

Subsequently, the selected colonies were spread on LB+ampicillin plates and cultured at 30° C. Then, a suitable amount of the cells were suspended in 2 ml of LB medium and cultured at 42° C. for 4 to 5 hours with shaking. The culture broth diluted 10⁵ times was inoculated on an LB plate. Among the obtained colonies, several hundreds of colonies were inoculated on an LB plate and an LB+ampicillin plate, and their growth was checked to confirm ampicillin susceptibility or resistance. Chromosomal DNA of an ampicillin-susceptible strain is deficient in a vector portion of pMANΔfis and the normal fis gene, which originally existed on the chromosomal DNA, or deletion-type fis gene. Several ampicillin susceptible strains were subjected to colony PCR to select strains in which the fis gene was replaced with the deletion-type gene as intended. Thus, a fis gene-disrupted strain, the MG1655Δfis strain, was obtained from the Escherichia coli MG1655.

(2) Disruption of hns Gene

An hns gene-disrupted strain was obtained from MG1655 in the same manner as in (1). The oligonucleotides of SEQ ID NOS: 5 and 6 (Primers 5 and 6) were synthesized as primers for amplifying a region of about 600 bp including about 40 bp at the N-terminus of the coding region of the hns gene and an upstream region of the same, and the oligonucleotides of SEQ ID NOS: 7 and 8 (Primers 7 and 8) were synthesized as primers for amplifying a region of about 600 bp including about 40 bp at the C-terminus of the coding region of the hns gene and a downstream region of the same.

Firs PCR was performed by using combinations of Primers 5 and 6 and Primers 7 and 8 and genomic DNA of the wild strain MG1655 prepared by a usual method as a template. Secondly, PCR was performed by using the obtained product of the first PCR as a template and Primers 5 and 8. A DNA fragment including the deletion-type hns gene constructed by the second PCR was obtained. The subsequent procedures were carried out in the same manner as in (1) to obtain an hns gene-disrupted strain MG1655Δhns.

(3) Disruption of dps Gene

A dps gene-disrupted strain was obtained from MG1655 in the same manner as in (1). The oligonucleotides of SEQ ID NOS: 9 and 10 (Primers 9 and 10) were synthesized as primers for amplifying a region of about 400 bp including about 20 bp at the N-terminus of the coding region of the dps gene and an upstream region of the same, and the oligonucleotides of SEQ ID NOS: 11 and 12 (Primers 11 and 12) were synthesized as primers for amplifying a region of about 300 bp including about 20 bp at the C-terminus of the coding region of the dps gene and a downstream region of the same.

Fist, PCR was performed by using combinations of Primers 9 and 10 and Primers 11 and 12 and genomic DNA of the wild stain MG1655 prepared by a usual method as a template. Secondly, PCR was performed by using the obtained product of the first PCR as a template and Primers 9 and 12. A DNA fragment including a deletion-type dps gene constructed by the second PCR was obtained. The subsequent procedures were carried out in the same manner as in (1) to obtain a dps gene-disrupted strain MG1655Δdps.

(4) Disruption of hupAB Gene

Since hupA and hupB are not adjacent to each other on the genome, these genes were disrupted separately. First, the hupA gene was disrupted. The oligonucleotides of SEQ ID NOS: 13 and 14 (Primers 13 and 14) were synthesized as primers for amplifying a region of about 300 bp including about 20 bp at the N-terminus of the coding region of the hupA gene and an upstream region of the same, and the oligonucleotides of SEQ ID NOS: 15 and 16 (Primers 15 and 16) were synthesized as primers for amplifying a region of about 300 bp including about 20 bp at the C-terminus of the coding region of the hupA gene and a downstream region of the same.

First, PCR was performed by using combinations of Primers 13 and 14 and Primers 15 and 16 and genomic DNA of the wild strain MG1655 prepared by a usual method as a template. Secondly, PCR was performed using the obtained product of the first PCR as a template and Primers 13 and 16. A DNA fragment including the deletion-type hupA gene constructed by the second PCR was obtained. The subsequent procedures were carried out in the same manner as in (1) to obtain a hula gene-disrupted strain MG1655ΔhupA

Subsequently, the hupB gene was disrupted in the hupA gene-disrupted strain MG1655ΔhupA. The oligonucleotides of SEQ ID NOS: 7 and 18(Primers 17 and 18)were synthesized as primers for amplifying a region of about 300 bp including about 20 bp at the N-terminus of the coding region of the hupB gene and an upstream region of the same, and the oligonucleotides of SEQ ID NOS: 19 and 20 (Primers 19 and 20) were synthesized as primers for amplifying a region of about 300 bp including about 20 bp at the C-terminus of the coding region of the hupB gene and a downstream region of the same.

First, PCR was performed by using combinations of Primers 17 and 18 and Primers 19 and 20 and genomic DNA of the wild strain MG1655 prepared by a usual method as a template. Secondly, PCR was performed by using the obtained product of the first PCR as a template and Primers 17 and 20. A DNA fragment including the deletion-type hupB gene constructed by the second PCR was obtained. The subsequent procedures were carried out in the same manner as in (1) to obtain a hupA gene and hupB gene-disrupted strain MG1655ΔhupAB.

(5) Culture of Gene-Disrupted Strains

The fis gene-disrupted strain MG1655Δfis, the hns gene-disrupted strain MG1655Δhns, the hupAB gene-disrupted strain MG1655ΔhupAB, the dps gene-disrupted strain MG1655Δdps and their parent strain MG1655 were cultured in a medium containing 20 mM NH₄Cl, 2 mM MgSO₄,40 mM NaHPO₄,30 mM KH₂PO₄, 0.01 mM CaCl₂, 0.01 mM FeSO₄, 0.01 mM MnSO₄, 5 mM citric acid, 10 mM glucose, 2 mM thiamine hydrochloride, 2.5 g/L casamino acid (Difco) and 50 mM MES-NaOH (pH 6.8) using a 10-ml volume L-tube. The amount of the culture broth at the start of the culture was 5 ml. The culture was performed at 37° C. with shaking by rotation at a rotation rate of 70 rpm. The medium, vessels and so forth were all subjected to autoclave sterilization before use.

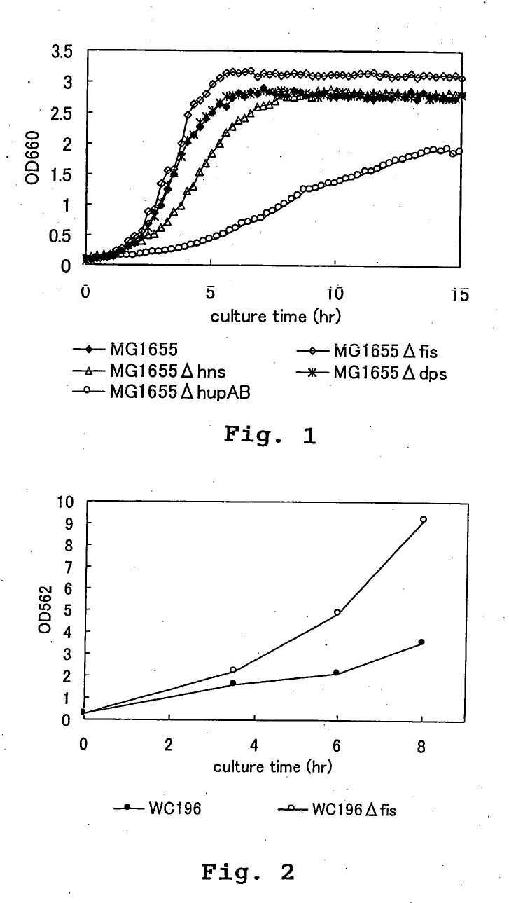

The cell concentration in the culture broth was measured over time. The cell concentration was determined by measuring turbidity at 660 nm using Biophotodetector (Advantech). The results are shown in FIG. 1.

As a result, it was observed that the dps gene-disrupted strain showed growth similar to that of the control strain, and the hns gene-disrupted strain and the hupAB gene-disrupted strain showed decreased growth. On the other hand, it was observed that thefts gene-disrupted strain showed growth improved as compared with that of the control strain. Thus, effect of disruption of the fis gene on fermentation production was verified.

### Example 2

### Disruption of Fis Gene of Escherichia Coli and its Effect on L-Lysine Production

(1) Disruption of fis Gene

A fis gene-disrupted strain WC196Δfis strain was obtained from the Escherichia coli WC196 strain in the same manner as in Example 1. The WC196 stain is an L-lysine producing bacterium derived from AEC resistant Escherichia coli. This strain was designated AJ13069 as a private number and deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) and received an accession number of FERM P-14690. Then, it was transferred to an international deposit under the provisions of the Budapest Treaty on Sep. 29, 1995, and received an accession number of FERM BP-5252 (see International Patent Publication WO96/17930). The L-lysine producing bacterium WC196 of Escherichia coli was transformed by using the plasmid for fis disruption obtained in Example 1, pMANΔfis. The subsequent procedures were carried out in the same manner as in Example 1 to obtain the fis gene-disrupted strain WC196Δfis from the L-lysine producing bacterium WC196 of Escherichia coli.

(2) Culture of fis Gene-Disrupted Strain

The fis gene-disrupted strain WC196Δfis and its parent strain WC196 were cultured in a medium containing 20 mM NH₄Cl, 2 mM MgSO₄, 40 mM NaHPO₄, 30 mM KH₂PO₄, 0.01 mM CaCl₂, 0.01 mM FeSO₄, 0.01 mM MnSO₄,5 mM citric acid, 50 mM glucose, 2 mM thiamine hydrochloride, 2.5 g/L casamino acid (Difco) and 50 mM MES-NaOH (pH 6.8) by using a 200-ml conical flask The amount of the culture broth at the start of the culture was 20 ml. The culture was performed at 37° C. with shaking by rotation at a rotation rate of 144 rpm. The medium, vessels and so forth were all subjected to autoclave sterilization before use.

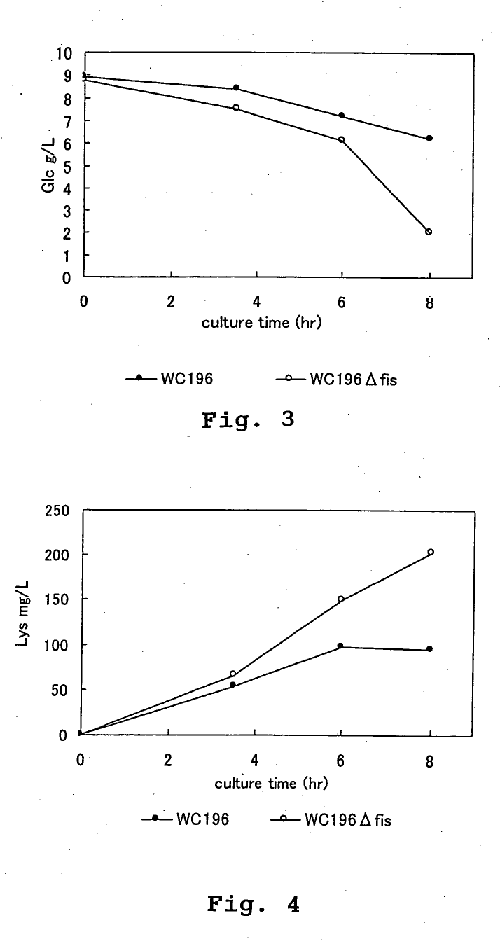

The cell concentration, glucose concentration and L-lysine accumulation in the culture broth were measured over time. The cell concentration was determined by measuring turbidity at 562 nm of the culture broth diluted with water to a suitable concentration using a spectrophotometer (Beckman). The glucose concentration and the L-lysine concentration were measured for the culture supernatant diluted to a suitable concentration after removal of the cells by centrifugation by using Biotech Analyzer (Sakura Seiki). The results are shown in FIGS. 2 to 4. Further, values of the L-lysine accumulation and the residual glucose concentration alter 8 hours of the culture are shown below.

TABLE 1
L-lysine accumulation and residual glucose concentration
after 8 hours for fis-disrupted strain

| | L-Lysine accumulation | |
|---|---|---|
| Bacterial strain | (mg/L) | Glucose (g/L) |
| WC196Δfis | 205 | 2.00 |
| WC196 | 95 | 6.20 |

As a result, it was observed that the fis gene-disrupted strain showed improvement as compared with the control strain with regards to all of the growth (FIG. 2), glucose consumption rate (FIG. 3) and L-lysine production rate (FIG. 4).

### Example 3

### Introduction of Plasmid for Producing L-Lysine into fis Gene-Disrupted Escherichia Coli Strain and its Effect on L-Lysine Production

(1) Introduction of Plasmid for Producing L-Lysine into fis Gene-Disrupted Strain

The fis gene-disrupted stain WC196Δfis obtained in Example 2 and its parent strain WC196 were transformed with plasmid pCABD2 (WO95/16042) containing a mutant dihydrodipicolinate synthetase gene, mutant aspartokinase III gene and dihydrodipicolinate reductase gene derived from Escherichia bacterium and a diaminopimelate dehydrogenase gene derived from Brevibacterium lactofermentum to obtain a WC196/pCABD2 strain and WC196Δfis/pCABD2 strain. The aforementioned mutant dihydrodipicolinate synthetase gene and mutant aspartokinase III gene both have a mutation for desensitizing the feedback inhibition by L-lysine.

(2) Culture of fis Gene-Disrupted Stain

The fis gene-disrupted WC196Δfis/pCABD2 strain and the wild type strain WC196/pCABD2 containing the fis gene were cultured in a medium containing 20 mM NH₄Cl, 2 mM MgSO₄, 40 mM NaHPO₄, 30 mM KH₂PO₄, 0.01 mM CaCl₂, 0.01 mM FeSO₄, 0.01 mM MnSO₄, 5 mM citric acid, 50 mM glucose, 2 mM thiamine hydrochloride, 2.5 g/L casamino acid (Difco) and 50 mM MES-NaOH (pH 6.8) by using a 200-ml conical flask. The amount of the culture broth at the start of the culture was 20 ml. The culture was performed at 37° C. with shaking by rotation at a rotation rate of 144 rpm. The medium, vessels and so forth were all subjected to autoclave sterilization before used.

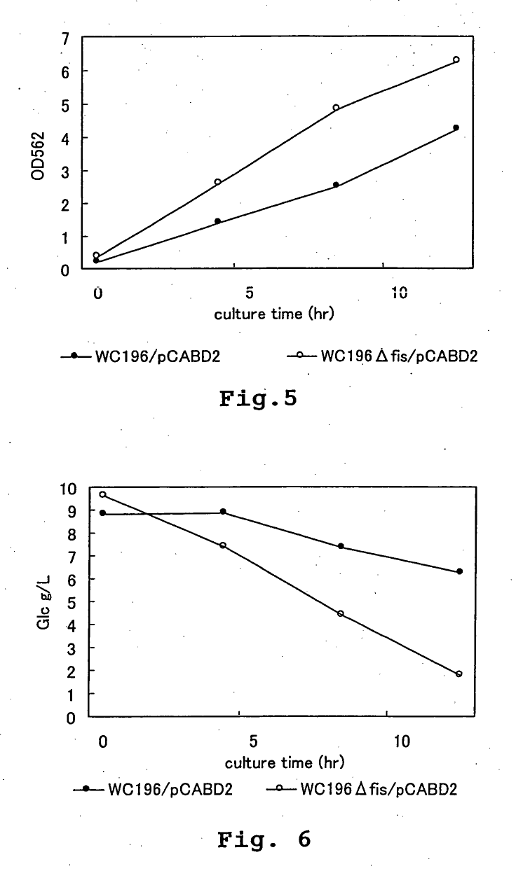

The cell concentration, glucose concentration and L-lysine accumulation in the culture broth were measured over time. The cell concentration was determined by measuring turbidity at 562 nm of the culture broth diluted with water to a suitable concentration using a spectrophotometer (Beckman). The glucose concentration and the L-lysine concentration were measured for the culture supernatant diluted to a suitable concentration after removal of the cells by centrifugation by using Biotech Analyzer (Sakura Seiki). The results are shown in FIGS. 5 to 7. Further, values of the L-lysine accumulation and the residual glucose concentration after 8 hours of the culture are shown below.

TABLE 2
L-lysine accumulation and residual glucose concentration
alter 8 hours for fis-disrupted strain

| | L-Lysine | |
|---|---|---|
| Bacterial strain | accumulation (g/L) | Glucose (g/L) |
| WC196/pCABD2 | 0.80 | 7.35 |
| WC196Δfis/pCABD2 | 1.60 | 4.40 |

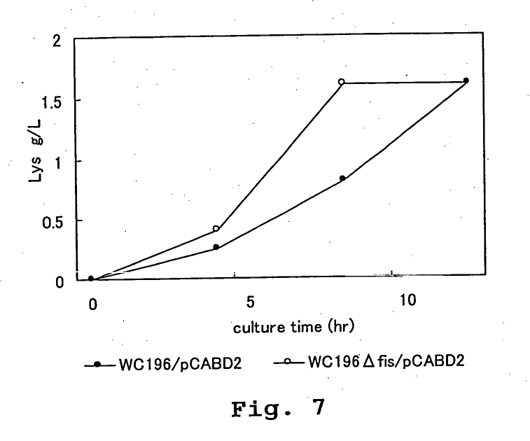

As a result, it was observed that the fis gene-disrupted strain into which the plasmid for L-lysine production was introduced was also improved as compared with the control strain with regard to overall growth (FIG. 5), glucose consumption rate (FIG. 6) and L-lysine production rate (FIG. 7).

While the invention has been described with reference to preferred embodiments thereof, it will be apparent to one skilled in the art that various changes can be made, and equivalents employed, without departing from the scope of the invention. Each of the aforementioned documents, including the foreign priority document JP2002-336340, is incorporated by reference herein in its entirety.

What is claimed is:

1. A method for producing a target substance using a bacterium belonging to the genus Escherichia comprising:
(a) culturing said bacterium in a medium resulting in production and accumulation of said target substance in said medium or said bacterium and
(b) collecting said target substance,
wherein said bacterium has an ability to produce said target substance, and FIS protein does not function normally in said bacterium.

2. The method of claim 1, wherein said fis gene has been disrupted so that it does not function normally.

3. The method of claim 1, wherein said bacterium belonging to the genus Escherichia is Escherichia coli.

4. The method of claim 1, wherein said target substance is selected from the group consisting of an L-amino acid and a protein.

5. The method of claim 4, wherein said target substance is an L-amino acid.

6. The method of claim 5, wherein said L-amino acid is L-lysine.

7. A method for producing an L-amino acid from the aspartic acid family using a bacterium belonging to the genus Escherichia comprising:
(a) culturing said bacterium in a medium resulting in production and accumulation of said L-amino acid in said medium or said bacterium and
(b) collecting said L-amino acid,
wherein said bacterium has an ability to produce said target substance, and FIS protein does not function normally in said bacterium.

8. The method of claim 7, wherein said aspartic acid family comprises lysine, threonine, and methionine.

9. The method of claim 7, wherein said fis gene has been disrupted so that it does not function normally.

10. The method of claim 7, wherein said bacterium belonging to the genus Escherichia is Escherichia coli.

11. A method for producing an amino acid selected from the group consisting of lysine, threonine, and methionine using a bacterium belonging to the genus Escherichia comprising:
(a) culturing said bacterium in a medium resulting in production and accumulation of said amino acid in said medium or said bacterium and
(b) collecting said amino acid,
wherein said bacterium has an ability to produce said target substance, and FIS protein does not function normally in said bacterium.

12. The method of claim 11, wherein said fis gene has been disrupted so that it does not function normally.

13. The method of claim 11, wherein said bacterium belonging to the genus Escherichia is Escherichia coli.

14. The method of claim 11, wherein said amino acid is lysine.


SEQUENCE LISTING (22 sequences)

SEQ ID NO: 1 - DNA, 20 bp, Artificial Sequence
  Description of Artificial Sequence primer for PCR
cctggatctttcgggaaatc

SEQ ID NO: 2 - DNA, 20 bp, Artificial Sequence
  Description of Artificial Sequence primer for PCR
tacgcgttgttcgaacatag

SEQ ID NO: 3 - DNA, 40 bp, Artificial Sequence
  Description of Artificial Sequence primer for PCR
ctatgttcgaacaacgcgtaaaatacggcatgaactaatt

SEQ ID NO: 4 - DNA, 20 bp, Artificial Sequence
  Description of Artificial Sequence primer for PCR
cactctgcaatcacttcaaa

SEQ ID NO: 5 - DNA, 20 bp, Artificial Sequence
  Description of Artificial Sequence primer for PCR
atagggaattctcgtaaaca

SEQ ID NO: 6 - DNA, 20 bp, Artificial Sequence
  Description of Artificial Sequence primer for PCR
tttaagtgcttcgctcattg

SEQ ID NO: 7 - DNA, 40 bp, Artificial Sequence
  Description of Artificial Sequence primer for PCR
caatgagcgaagcacttaaattcctgatcaagcaataatc

SEQ ID NO: 8 - DNA, 20 bp, Artificial Sequence
  Description of Artificial Sequence primer for PCR
agaaacggtggaagcctatc

SEQ ID NO: 9 - DNA, 20 bp, Artificial Sequence
  Description of Artificial Sequence primer for PCR
cccatacagctactggcgct

SEQ ID NO: 10 - DNA, 20 bp, Artificial Sequence
  Description of Artificial Sequence primer for PCR
taatttagcggtactcataa

SEQ ID NO: 11 - DNA, 42 bp, Artificial Sequence
  Description of Artificial Sequence primer for PCR
ttatgagtaccgctaaattagagtctaacatcgaataaatcc

SEQ ID NO: 12 - DNA, 20 bp, Artificial Sequence
  Description of Artificial Sequence primer for PCR
cggcaaaaacgtgatgcacg

SEQ ID NO: 13 - DNA, 20 bp, Artificial Sequence
  Description of Artificial Sequence primer for PCR
atattccgacttttagctga

SEQ ID NO: 14 - DNA, 20 bp, Artificial Sequence
  Description of Artificial Sequence primer for PCR
cagttgagtcttgttcataa

SEQ ID NO: 15 - DNA, 40 bp, Artificial Sequence
  Description of Artificial Sequence primer for PCR
ttatgaacaagactcaactgaaagacgcagttaagtaaga

SEQ ID NO: 16 - DNA, 20 bp, Artificial Sequence
  Description of Artificial Sequence primer for PCR
aacattgatattgatgagcg

SEQ ID NO: 17 - DNA, 20 bp, Artificial Sequence
  Description of Artificial Sequence primer for PCR
tggacattcatcctgtgaag

SEQ ID NO: 18 - DNA, 20 bp, Artificial Sequence
  Description of Artificial Sequence primer for PCR
caattgagatttattcactc

SEQ ID NO: 19 - DNA, 40 bp, Artificial Sequence
  Description of Artificial Sequence primer for PCR
gagtgaataaatctcaattgaaagacgcggtaaactaagc

SEQ ID NO: 20 - DNA, 20 bp, Artificial Sequence
  Description of Artificial Sequence primer for PCR
cgccaatcaggtaaccactc

SEQ ID NO: 21 - DNA, 497 bp, Escherichia coli
  CDS (101)..(397)
cgcacattcaacgccattgaggatgccagcgaacagctggaggcgttggaggcatacttcgaaaattttgcgtaaacagaaataaagagctgacagaactatgttcgaacaacgcgtaaattctgacgtactgaccgtttctaccgttaactctcaggatcaggtaacccaaaaacccctgcgtgactcggttaaacaggcactgaagaactattttgctcaactgaatggtcaggatgtgaatgacctctatgagctggtactggctgaagtagaacagcccctgttggacatggtgatgcaatacacccgtggtaaccagacccgtgctgcgctgatgatgggcatcaaccgtggtacgctgcgtaaaaaattgaaaaaatacggcatgaactaattcaggttagctaaatgcttgattaaaaaggcgctactcggcatggggaagcgccttttttataggtgtcacaaagggagtgaccatgagaacaggatgt

SEQ ID NO: 22 - protein, 98 aa, Escherichia coli [sequence omitted]